(19) United States
(12) Patent Application Publication
(10) Pub. No.: US 2004/0136864 A1
(43) Pub. Date: Jul. 15, 2004

(54) Treatment of dentures at elevated pressure

(76) Inventor: William L. Barham, Mt. Airy, NC (US)

Correspondence Address: Robert W. Pitts, PO Box 11483, Winston-Salem, NC, 27116-1483, US

(21) Appl. No.: 10/339,802

(22) Filed: Jan. 10, 2003

(51) Int. Cl. A61L 2/08; A61L 9/00; B01J 19/00; B01D 11/04; B01D 11/02; A61L 2/00

(52) U.S. Cl. 422/27; 422/1; 422/28; 422/32; 422/33; 422/40; 422/256; 422/261; 422/295; 422/297; 422/298; 422/300; 422/305; 422/307

(57) ABSTRACT

An apparatus and methods for treating dentures at elevated pressured to enhance the effectiveness of the treatment employs a sealed pressure containment vessel. Denture cleaning, disinfecting, deorderizing, brightening, bleaching can be performed using this apparatus and method and the effectiveness of conventional active agents in removing plaque or the biolayer on the surface of the denture can be improved. Active agents can be forced into pores, fissures and microscopic openings in dentures to remove pathogens or contaminants that cannot be reached by conventional processes. The effectiveness of conventional effervescent denture treatment tablets can also be increased.

### BACKGROUND OF THE INVENTION

1. Field of the Invention

This invention relates to treating, cleaning, disinfecting and brightening dentures by the use of a pressure containment vessel in which a denture treatment, such as cleaning, disinfecting, or brightening agents, is introduced into water in the pressure containment vessel. This invention is also related to the use of a foaming agent to increase the pressure in the pressure containment vessel to enhance the action of the cleaning, disinfecting, or brightening agents on a denture disposed in a cavity in the pressure containment vessel.

2. Description of the Prior Art

Many methods and techniques have been suggested to facilitate cleaning and disinfecting dentures. Denture cleaning can be broadly defined as the reduction of a biolayer or biofilm and plaque containing bacteria, fungi and virus and pathogens or debris in general. Generally some method is employed to disrupt the plaque coating or biolayer and to soak the denture to allow chemical solutions to interface with denture surface and with porosities, micro-channels, fissures, cracks, fractures and spaces between teeth and the acrylic that retains the teeth in the gum portion of the denture. Additional pores, micro-channels, fissures, cracks, and fractures are formed as a denture ages.

One common approach to cleaning dentures is to use effervescent tablets, which foam when placed in water. Conventional tablets contain cleaning agents. Traditionally, these tablets have a composition containing a variety of sulfate salts, such as bisulfates, monopersulfates and sulfates acting as detergents, oxidizers and the like. They have also used alkali metal and alkaline earth metal halides as bleaches. Such compositions have also included perborate, carbonate and phosphate slats in varying amounts to provide effervescence and activation. A discussion of some of these traditional effervescent cleaning compositions can be found in U.S. Pat. No. 4,857,224, which is incorporated herein be reference.

Limitations have been encountered with standard prior art methods. Strong solutions containing alcohol adversely affect the acrylic. Some cleaning or treatment solutions are too strong for biocompatibility with oral tissues. Microwaving weakens the dentures and may warp the acrylic due to uneven heat buildup. Mechanical means to scrap the denture surface are incomplete and the size of practical mechanical scraping means is too large to remove plaque in microscopic pores, micro-channels, fissures, cracks, and fractures. Furthermore mechanical means tend to scarify or abrade the denture surface thereby increasing fissures where pathogens may build up.

### SUMMARY OF THE INVENTION

It has been apparent for some time that the plaque or biolayer must first be perforated and removed from the denture surface in order to effectively disinfect a denture. Any system that can attack the plaque or biolayer on large as well as small surfaces would represent an improvement. Experimentation with a new method of cleaning a denture under pressure has produced a noticeable difference in dentures. Denture wearers have noticed that dentures cleaned in this manner feel as if a layer of slime has been removed from the denture and that the denture feels cleaner than with other conventional methods. This noticeable improvement is believed to be due to the removal of a larger proportion of the plaque or biolayer that builds up on a denture.

A denture treating apparatus in accordance with this invention comprises a sealed pressure containment vessel. The pressure containment vessel includes a cavity having a volume sufficient for at least one denture, a denture treatment, such as an effervescent cleaing tablet and sufficient water with which the denture treatment reacts to release a foam and an active denture treating agent. The seal holds pressure within the pressure containment vessel to permit release of foam when the denture treatment is introduced into the water. An elevated pressure within the pressure containment vessel will thus enhance removal of contaminants and pathogens from the denture. Pressure relief means in the form of a seal only capable of providing sealing integrity up to a prescribed pressure, less than the pressure that can be developed in the pressure containment vessel, or a separate pressure relief valve, could also be employed.

This pressure containment vessel can then be used in a method of cleaning or treating a dental prosthesis. Typically in the first step in this method the dental prosthesis is immersed in water in a pressure containment vessel. A cleaning or treating agent is then added to the water. A foaming agent is also added. Both the cleaning or treating agent and the foaming agent could be included in a single tablet, or they could be separately added in tablet or powder form. The next step involves sealing the pressure containment vessel to elevate pressure in the pressure containment vessel as the foaming agent reacts with the water so that the cleaning agent acts on the denture at a pressure in excess of atmospheric pressure. Other treatments, including disinfecting, deorderizing, brightening, or bleaching could also be performed using this method. Multiple treatments can be carried out at the same time by introducing multiple active agents into the pressure containment vessel. Devices suitable for home use and for professional dental use could include the same basic steps and similar components.

This invention also involves a method of removing a biolayer from the exterior of a dental prosthesis. This process too would start by immersing the dental prosthesis in water in a pressure containment vessel. An active agent would then be to the water followed by addition of a foaming agent. The pressure containment vessel would then be sealed to elevate pressure in the pressure containment vessel as the foaming agent reacts with the water so that the active agent acts on the denture at a pressure in excess of atmospheric pressure.

### BRIEF DESCRIPTION OF THE DRAWINGS

FIG. 1 is a schematic view of a small section of a denture showing small pores, cracks, fissure and minute openings in the denture, especially the type that can form as the denture ages.

FIG. 2 is an exploded view of the pressure containment vessel of a first embodiment of this invention.

FIG. 3 is a schematic view of the pressure containment vessel of FIG. 1, showing a denture and a denture treatment tablet dispersed in warm water filling the pressure containment cavity.

FIG. 4 is an enlarged view showing the seal and mating threads of the cover and base which form the pressure containment vessel of FIGS. 2 and 3.

FIG. 5 is an exploded section view of the pressure containment vessel of FIGS. 2-4.

FIG. 6 is a section view, similar to FIG. 5, showing the cover mated to the base to seal the pressure containment cavity.

FIG. 7 is a top view of the cover of the embodiment of FIGS. 2-6, showing the circular shape of the seal groove and the cover threads.

FIG. 8 is a bottom view of the cover shown in FIG. 7.

FIG. 9 is a view of a second embodiment of a pressure containment vessel in accordance with this invention, in which the cover is attached to the base by camming surfaces instead of threads.

FIG. 10 is a view of a third embodiment of this invention in which a ball check valve is used to release pressure in the pressure containment vessel after a specified pressure, greater than atmospheric pressure, has been developed.

FIG. 11 is a view of a fourth embodiment of this invention in which a conical seal is used and a pressure release means.

### DETAILED DESCRIPTION OF THE PREFERRED EMBODIMENTS

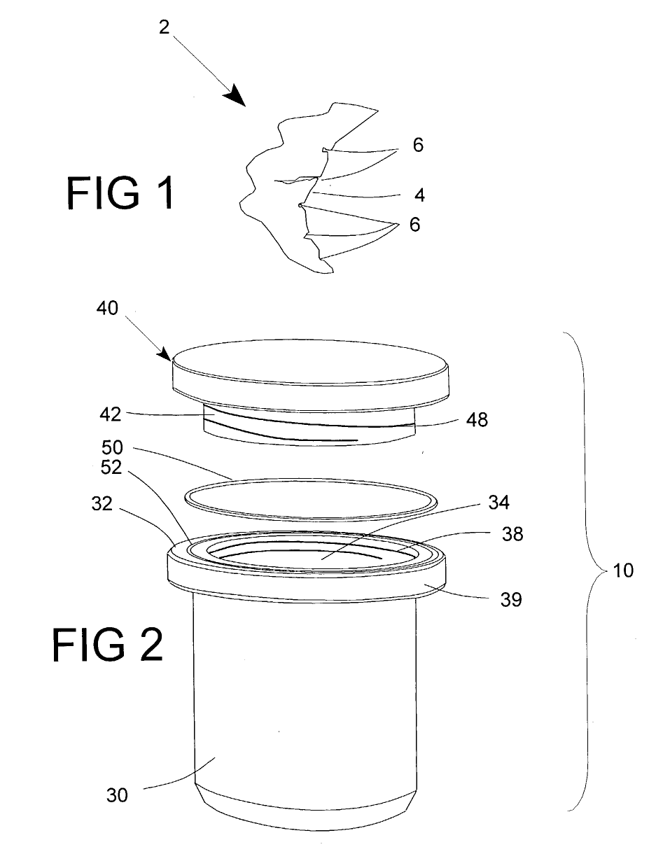

Dentures typically have a number of artificial teeth bonded to an acrylic base. The acrylic base, which forms the gum portion of the denture, will have a characteristic number of pore or small openings extending into the surface, as is typical for any polymeric surface. However, as the dentures age, the porosity of the acrylic base increases and additional pores, fissures, cracks, channels and microscopic indentations or openings are formed in the denture. FIG. 1 is a schematic view of a small portion of a denture 2 showing a plurality of pores, fissures, cracks, channels and microscopic indentations or openings 6 extending into an interior surface 4 of a denture 2. Although not shown, a biolayer or plaque will also build up on the exposed surface 4, and in or over the pores, fissures, cracks, channels and microscopic indentations or openings 6. Since the biolayer or plaque is difficult to remove when placed in an effervescent denture treatment or cleaning solution, it is also difficult to remove pathogens that may have collected within these pores, fissures, cracks, channels and microscopic indentations or openings 6. Furthermore the cleaning or treating solution at atmospheric pressure cannot be expected to enter into closed pores because of the pressure of the gas or air trapped in these closed pores. Since the air or gas would be compressed by fluid attempting to enter closed pores initially at atmospheric pressure, less of the surface of the pores, fissures, cracks, channels and microscopic indentations or openings would be wetted by the cleaning or denture treatment solutions at atmospheric pressure. Since pathogens, such as bacteria, fungi and viruses would be expected to inhabit these pores and small openings, a conventional effervescent cleaning composition would not be effected to remove these pathogens from closed pores and similar structures, commonly found in dentures. In this invention, an increase in pressure will enhance penetration of active agents into these micro-spaces and thereby kill, destroy or neutralize additional pathogens, and order causing bacteria.

The bonding of the base plate acrylic to denture teeth is incomplete. Very small spaces and cracks therefore are commonly formed between teeth and baseplate. These mmicrospaces are ideal areas to enhance the growth of bacterial, fungal or viral pathogens. Here to, atmospheric pressure will not push the active chemistry or agents into these areas even if the surface coating of biofilm is removed. The increased overpressure after penetrating and lifting the biolayer with the detergent chemistry continues to push the disinfecting chemistry much deeper into the micro-spaces, fissures and cracks than with passive solutions at atmospheric pressure.

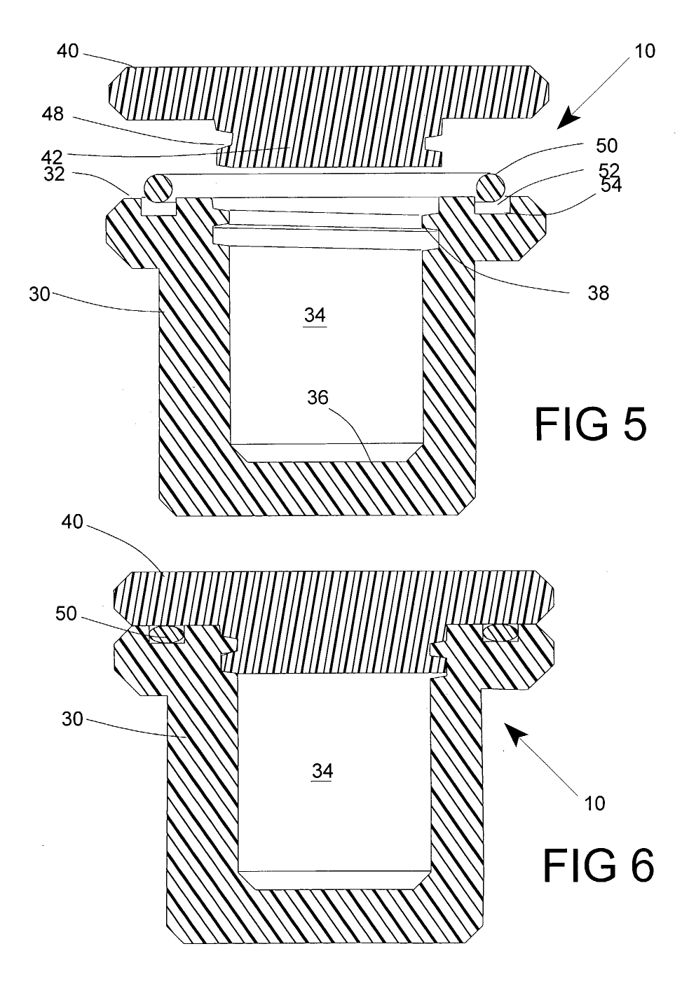

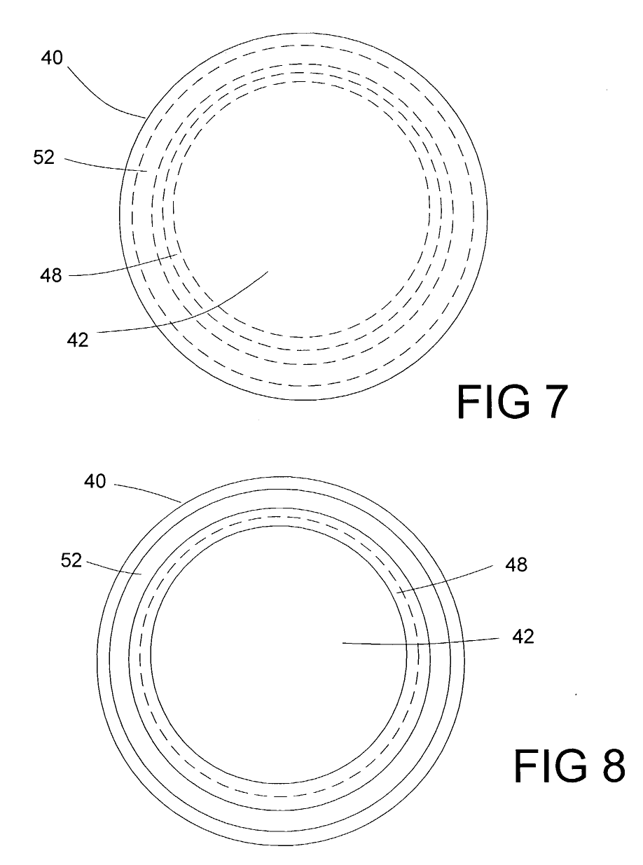

One embodiment of a pressure containment vessel 10 is shown in FIGS. 2-8. Pressure containment vessel 10 can be used with an effervescent denture cleaning or treatment tablet 70, and will improve the effectiveness of a conventional effervescent denture cleaning or treatment composition. Vessel 10 includes a base 30 and a cover 40 that can be assembled to from a cavity 34 in which a denture can be placed. A seal 50, here in the form of an elastomeric O-ring, can be positioned within an annular seal groove 52 that extends into the top surface 32 of a flange 39 on the top of base 30. When the cover 40 is screwed to the base 30, the seal 50 is compressed to at least initially prevent escape of fluid, either in the form of a liquid or a gas, from the cavity 34. The cover 40 has a cylindrical boss 42 extending from its lower surface and helical threads 48 on the exterior of the boss 42 engage interior threads 38 at the top of the cavity 34. The mating threads 38 and 48 are not intended to form a sealing surface, and pressurized fluid in the form of a gas or a liquid can pass through the mating threads 38 and 48. A relief groove can even extend through the treads 35 and 48 on either the boss or cavity sidewall or both to permit passage of a fluid so that the seal 50 can be the only means for establishing sealing integrity. The mating threads 38 and 48 are, however, relied upon to compress the seal 50 against the lower surface of the seal groove and against the bottom surface of the cover 40 extending beyond the boss 42 sufficient so that an elevated or gauge pressure of at least a first level can be maintained in the cavity 34. FIG. 4 shows a compressed seal 50 and clearance that could be expected between mating threads 38 and 48. Comparison of FIGS. 5 and 6 demonstrates the manner in which an O-ring seal, having a circular cross section, will be compressed between the cover 40 and the base 30.

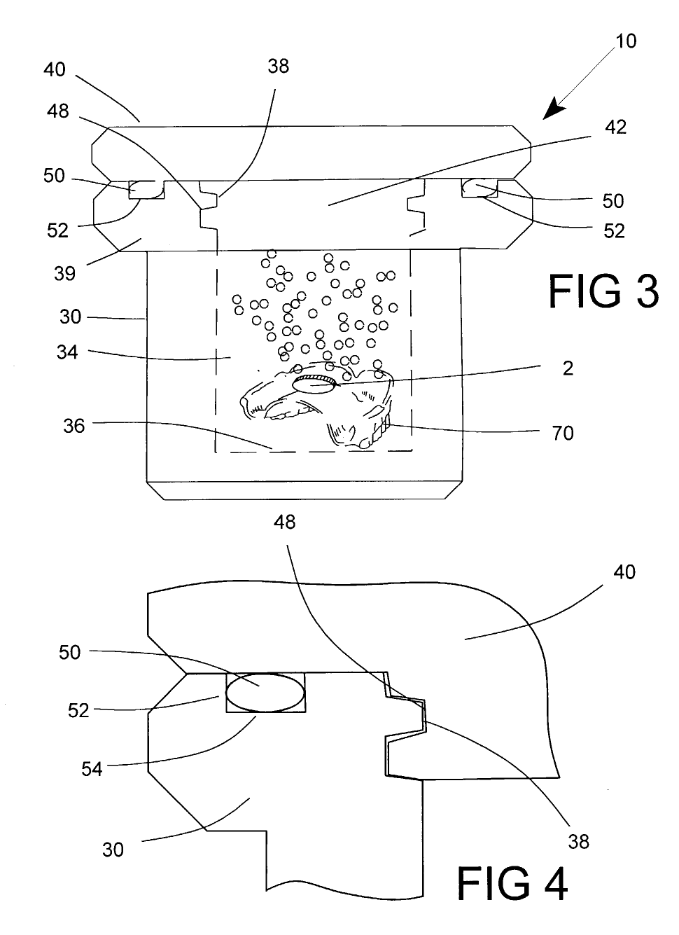

FIG. 3 depicts the manner in which an elevated pressure can be generated in cavity 34. A denture 2 can be placed within the cavity 34, resting on the bottom cavity surface 36. This cavity is then filled with warm water. One or more effervescent denture cleaning or treatment tablets 70 can then be placed in the water within the cavity. The cover 40 can then be screwed onto the base 30. In this embodiment, the boss 42 enters the top of the cavity 34. Excess water can flow out of the top of the cavity 34 as the cover 40 is screwed on. When the cover 40 is tightly secured to the base 30, and the seal 50 is compressed to seal the cavity 34, the entire volume of the cavity 34 will be filled leaving a minimum air space within the cavity 34. However, the effervescent tablet 70 will react with the water and gas will be released to pressurize the fluid within the cavity 34. It has been found that a gauge pressure of 24 psi, or 24 psi above atmospheric pressure, can be achieved by introduction of two standard effervescent tablets in a pressure vessel cavity 34 having a volume of approximately 225 cu. cm. The seal 50 should be capable of holding pressure in the containment vessel for at least four to five hours, and preferably overnight, for this application. A single effervescent tablet introduced in a container of the same size increase the pressure with the containment vessel cavity to approximately 10 psi above atmospheric. Based on user reaction, it is believe that this elevated pressure enhances the ability of the active ingredient or agent in the effervescent table to remove plaque or the biolayer on the denture. The high pressure bubbles released by the foaming agent in the tablet can also be responsible for first removing plaque or the biolayer and then penetrating into micro-spaces to eliminate other contaminants or pathogens from the denture.

The O-ring seal 50 not only seals the cavity 34 to allow the pressure to build up, but also can provide a pressure relief means to prevent excessive pressure from building up. The seal can be designed so that it only holds pressure up to a specified limit, after which it allows pressurized fluid to escape from the cavity 34.

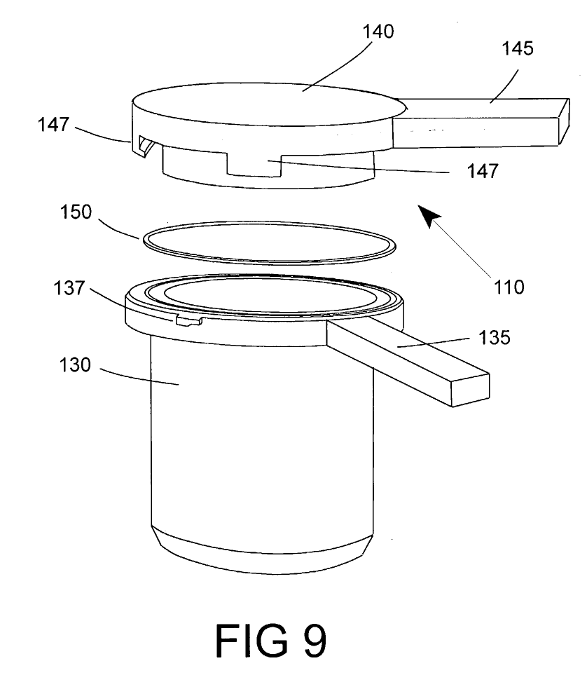

Another embodiment of a pressure containment vessel is shown in FIG. 9. This embodiment includes a handle 145 extending from one side of a cover 140 as well as a second handle 135 extending from base 130. A plurality of latches 147 extend downwardly from the peripheral edges of the cover 140. These latches 147 have an L-shaped profile with a lip projecting inwardly from the bottom of each latch 147. These latches 147 engage a similar number of locking wedges 137 extending from the periphery of the base 130. Each locking wedge 137 has a stepped lower surface with an inclined surface located between a shallow surface and a deeper locking surface. The lower surface of each wedge 137 thus serves as a camming means so that as the cover 140 is rotated relative to the base 130, the lip on the corresponding latch 147 rides along this stepped surface to bring the cover 140 into tight engagement with the base 130. When the handles 145 and 135 are rotated into alignment, the cover 140 will move toward the base 130 to compress the seal 150. The latches 147 and the locking wedges 137 replace the threads on the embodiment of FIGS. 2-8, but the mechanical advantage provided by these handles make it easier to close and then open the pressure containment vessel 110. Less force will be required, which can be a significant advantage for older users of this device. As in the embodiment of FIGS. 2-8, the O-ring seal 150 can function both as a means for initially withstanding the pressure developed by the release of a foaming agent upon introduction of an effervescent tablet into water in a pressure containment cavity and as a release for excess pressure. The latches 147 and the locking wedges 137 can also permit gradual release of pressure. If the latches 147 are partially released from the wedges 137, but the L-shaped portions of latches 147 still overlap the shallower sections of wedges 137, the compression of seal 150 will be reduced allowing the gradual escape of pressure before the cover 140 is completely released from the base 130.

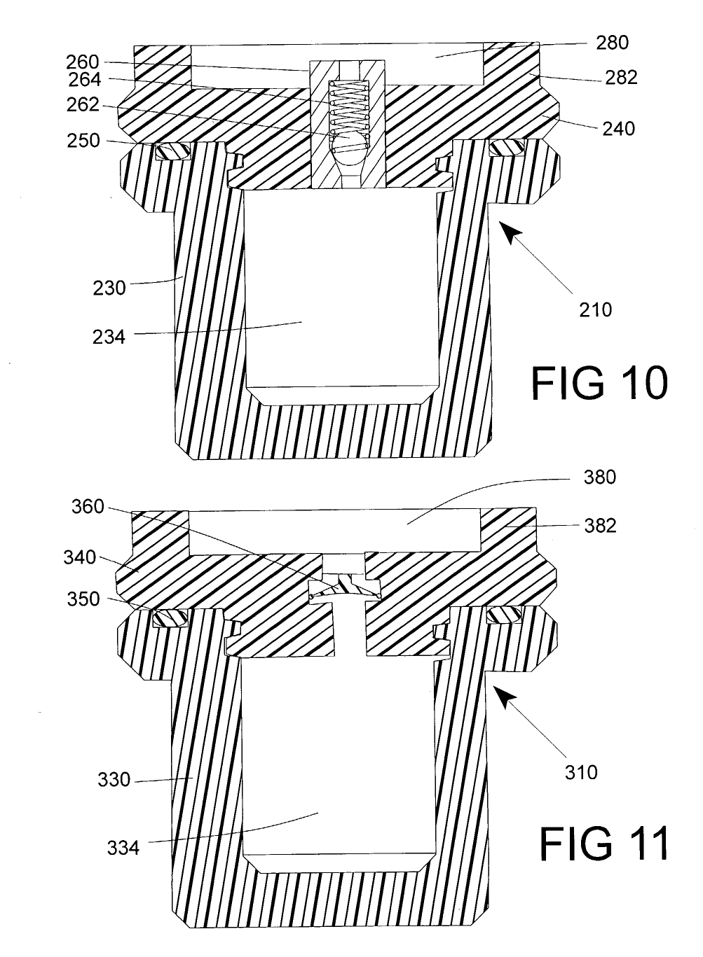

FIGS. 10 and 11 show two additional embodiments of pressure containment vessels in which a pressure release means other than the O-ring seal is provided. In FIG. 10, a mechanical check valve 260 extends through the top of the cover 240. This check 260 valve includes a ball 262 held against a seat by a coil spring 264. When the pressure within cavity 234, formed in base 230, reaches a specified level, greater than atmospheric pressure, the force on the ball will be sufficient to overcome the force exerted by the coil spring 264. Pressurized fluid in the cavity 234 can then be released. The pressure vessel 210 also includes an overflow reservoir 280 on the top of the cover 240. This reservoir is formed by a peripheral ledge 282 surrounding the opening of the check valve 260. In this embodiment the O-ring seal 250, when compressed between the cover 240 and the base 230, will be able to withstand a greater pressure than the pressure relief check valve 260.

FIG. 11 shows a similar arrangement with a pressure relief valve 360 located at the center of the cover 340 of this alternate pressure containment vessel 310. This valve includes a conical elastomeric member 360 located within a passage. When a specified internal pressure is developed within cavity 334, the peripheral wedges of the conical elastomeric member 360 will be deflected sufficiently to allow the escape of pressurized fluid. The elastomeric member 360 can be held in place with its opening by ribs extending across its central section, so that only the peripheral edges can move, thus permitting this valve to open and function as a pressure relief valve. As in the embodiment of FIG. 10, an overflow reservoir 380 is formed by peripheral walls 382 on the top of the cover 340. The cover 340 is screwed onto the base 330 to compress the seal 350 in the same manner as with other embodiments. The pressure relief valves shown in FIGS. 10 and 11 are only intended to be examples of valve configurations that can be used to relieve pressure before the cover is separated from the base. Other configurations can be employed. For example, a manually operated pressure relief valve could also be employed. The O-ring seals used in each embodiment are also merely representative of sealing means that could be employed. Gaskets could also be employed. Threads with an interference fit could also be employed, especially when a separate pressure relief means is employed as in the embodiment of FIGS. 10 and 11. A pressure gauge could also be added to permit a user to determine the precise pressure in the pressure containment vessel. Incorporation of such a gauge may not be necessary for home use of this device for conventional denture cleaning, but more sophisticated treatments that could be performed in a dental office or laboratory may require pressure monitoring, at least to insure that a sufficient maximum pressure has been developed within the pressure containment cavity.

A simple home cleaning version of this invention would normally employ conventional effervescent denture cleaning tablets including both cleaning and foaming agents. Conventional tablets, such as Efferdent denture cleaning tablets and Polident denture cleaning tablets could be employed. Efferdent is a trademark of Warner-Lambert and Polident is a trademark of Glaxo Smith Kline. Use of this pressure enhanced denture treatment process is not limited to use of these commercially available compositions. Other treatments, including cleaning, disinfecting, deodorizing, brightening, bleaching or other compositions could also be employed. For example, the treatment agent could include menthol or eucalyptus oil. Other treatment agents could include, but would not be limited to, cetylpyridinum chloride, chlorhexidiene gluconate, eugenol, clove oil or peppermint oil. Chlorine dioxide could be used as the active agent and as the foaming agent for denture treatment in a dentist's office. As this compound dissociates the chlorine would provide the anti-bacterial agent and the oxygen would increase the pressure within the pressure containment vessel. Other simple foaming agents could also be employed. For example, baking soda and a salt could be used to make the water acidic to release carbon dioxide. In other words, both the materials and the pressure containment structures disclosed herein are merely intended to be representative, and other compositions and mechanical components would be readily apparent to one of ordinary skill in the art. Therefore this invention is not limited to the representative embodiments shown herein, but is instead defined by the following claims.

I claim:

1. A denture treating apparatus comprising:
a pressure containment vessel including a cavity having a volume sufficient for at least one denture, a denture treatment and sufficient water with which the denture treatment reacts to release a foam and an active denture treating agent, and
a seal for sealing the pressure containment vessel for a sufficient time for the release of foam when the denture treatment is introduced into the water to develop an elevated pressure within the pressure containment vessel to enhance removal of contaminants and pathogens from the denture.

2. The denture treating apparatus of claim 1 including means for releasing pressure when the pressure within the pressure containment vessel reaches a specified level in excess of atmospheric pressure.

31. The denture treating apparatus of claim 2 wherein the means for releasing pressure comprises a valve.

4. The denture treating apparatus of claim 2 wherein the means for releasing pressure comprises the seal and surfaces of the pressure containment vessel surrounding the seal.

5. The denture treating apparatus of claim 4 wherein the seal comprises an O-ring seal.

6. The denture treating apparatus of claim 2 wherein the means for releasing pressure comprises means for releasing pressure of about 24 psi relative to atmospheric pressure.

7. The denture treating apparatus of claim 1 wherein the pressure containment vessel includes a cavity having a cross sectional area at least equal to the maximum cross sectional area of a denture.

8. The denture treating apparatus of claim 1 wherein the pressure containment vessel comprises a base having a cavity extending inwardly from one face and a cover securable to the base to close the cavity.

9. The denture treating apparatus of claim 8 wherein the cover includes a boss projecting partially into the cavity so that excess water can be expelled from the cavity as the cover is assembled to the base so that the cavity is completely filled without air voids when the cover is completely assembled to the base and the cavity is sealed.

10. The denture treating apparatus of claim 1 wherein the pressure containment vessel and the seal maintain a pressure sufficient to force the treating agent into pores and fissures in the denture.

11. A method of treating a dental prosthesis comprising the steps of:
immersing the dental prosthesis in water in a pressure containment vessel;
adding a treating agent to the water;
adding a foaming agent to the water;
sealing the pressure containment vessel to elevate pressure in the pressure containment vessel as the foaming agent reacts with the water so that the treating agent acts on the denture at a pressure in excess of atmospheric pressure.

12. The method of claim 11 wherein the treating agent comprises a denture cleaner.

13. The denture treating apparatus of claim 601 wherein the treating agent comprises a disinfectant.

14. The method of claim 11 wherein the treating agent comprises a brightening agent.

15. The method of claim 11 wherein the treating agent comprises a bleaching agent.

16. The method of claim 11 wherein the treating agent comprises an anti-fungal agent

17. The method of claim 11 wherein the treating agent and the foaming agent are included in a tablet that is dropped into the water prior to sealing the pressure containment vessel.

18. The method of claim 11 including the step of releasing pressure in the pressure containment vessel prior to opening the pressure containment vessel.

19. The method of claim 11 including the step of completely filling the pressure containment vessel.

20. The method of claim 11 wherein an elevated pressure is maintained in the pressure containment vessel for a time sufficient for the cleaning agent to enter into pores, cracks, fissures and minute openings in the dental prosthesis into which the cleaning agent cannot enter at atmospheric pressure.

21. A method of removing a biolayer from the exterior of a dental prosthesis comprising the steps of:
immersing the dental prosthesis in water in a pressure containment vessel;
adding an active agent to the water;
adding a foaming agent to the water;
sealing the pressure containment vessel to elevate pressure in the pressure containment vessel as the foaming agent reacts with the water so that the active agent acts on the denture at a pressure in excess of atmospheric pressure to more effectively remove the external biolayer.

22. The method of claim 21 wherein the active agent and the foaming agent are included in a tablet that is dropped into the water prior to sealing the pressure containment vessel.

23. The method of claim 21 comprises the further step of maintaining an elevated pressure after the external biolayer is removed so that an active agent can penetrate fissures in the denture.

24. The method of claim 23 wherein an active disinfecting agent penetrates fissures in the denture under the influence of elevated pressure.

25. The method of claim 23 wherein an active deodorizing agent penetrates fissures in the denture under the influence of elevated pressure.
